(19) United States
(12) Patent Application Publication
(10) Pub. No.: US 2007/0219565 A1
(43) Pub. Date: Sep. 20, 2007

(54) Kinetic anchoring deployment system

(76) Inventor: Vahid Saadat, Saratoga, CA (US)

Correspondence Address: LEVINE BAGADE HAN LLP, 2483 EAST BAYSHORE ROAD, SUITE 100, PALO ALTO, CA, 94303, US

(21) Appl. No.: 11/378,218

(22) Filed: Mar. 17, 2006

(51) Int. Cl. A61B 17/10

(52) U.S. Cl. 606/142

(57) ABSTRACT

A kinetic anchoring deployment system utilizing a high-impulse, high-velocity anchor launching mechanism is described herein. The assembly generally has a handle, a flexible elongate body, and a launch assembly thereupon. The launch assembly uses a number of different mechanisms for creating a high-impulse shock wave for launching a carriage carrying a tissue anchor, e.g., combustible materials, rapid vaporization of a fluid, hydraulic energy transmission, laser energy, compression springs, electromagnetic energy, etc. The deployment system may be advanced intravascularly and/or intraluminally within a patient body for treating a number of different indications.

### FIELD OF THE INVENTION

The present invention relates generally to medical devices used for intravascular or intraluminal anchor placement within a body. More particularly, the present invention relates to apparatus and methods for intravascularly and/or intraluminally deploying tissue anchors or for rapidly piercing tissue regions, for instance, for taking biopsy samples within a body utilizing a rapid anchor delivery system.

### BACKGROUND OF THE INVENTION

The treatment of tissue within a body is generally made difficult especially when instruments are advanced and positioned via an intravascular or intraluminal approach. For instance, procedures which require access within the chambers of a patient's heart typically involve introducing a flexible catheter through a percutaneous incision and threading the catheter through the patient's vasculature until access to the appropriate chamber is acquired.

However, once within the heart chamber, a procedure such as delivering and deploying tissue anchors from the catheter into the heart tissue is made difficult by factors such as the tissue anatomy, resistance, as well as limitations of force transmission along the catheter shaft from the user to the catheter tip.

The tissue walls, especially within the heart, are usually thin and pliable thus making it difficult to exert any tissue anchoring force upon them. Intravascular instruments which utilize a torquing motion, such as cardiac lead implantation anchors, requires the transmission of a torque along the entire length of the catheter and may cause wrapping of the tissue around the instrument.

Other procedures which may require the pushing of a needle into the tissue wall may also inadvertently force the tissue to tent out relative to the needle and also potentially injure adjacently located organs or other anatomical structures behind the tissue. An example of a procedure of this type is a biopsy gun. A spring loaded biopsy gun, such as the Gallini ABS® reusable automatic high-speed biopsy gun (Gallini Medical SRL, Mantova, Italy) shoots a biopsy needle into the tissue at a rapid rate to both minimize sensation of pain and to also pierce hard tissues such as tumors. Heretofore, such biopsy guns have had rigid short shafts which are unable to bend or reach distal tissues in the body.

Thus, an instrument which may be advanced intravascularly or intraluminally and then deploy one or more tissue anchors directly into the tissue is desirable.

### BRIEF SUMMARY OF THE INVENTION

An anchor delivery assembly may generally have a handle assembly and a flexible elongate body extending from the handle. The flexible elongate body is sufficiently flexible to be advanced within the patient through the vasculature or intraluminally and has an anchor launch assembly positioned upon the distal end of the elongate body. The anchor launch assembly may house one or more tissue anchors which may be deployed or ejected at high speed through a distal opening defined in the distal end of the anchor launch assembly.

Despite the low mass of the tissue anchor relative to the underlying tissue resistance and also despite the inherent flexibility of the elongate body, a tissue anchor may be launched rapidly from the launch assembly within a short launch time to generate high kinetic energy and permits the generation of a high-velocity anchor having a large impulse to facilitate the insertion of the tissue anchor into the underlying tissue. In another alternative, rather than firing tissue anchors, the high kinetic energy launch assembly may be utilized to launch, e.g., a coring needle positioned near or at the distal end of a flexible member, to obtain biopsy tissue samples.

One mechanism for generating a high-velocity high-impulse anchor may utilize a spark generator positioned upon the end of a catheter for creating an explosive shock wave, typically used in intracorporeal lithotripsy instruments. The spark generating assembly may be connected via one or more wires routed through the elongate body to an ignition system or power supply located outside the patient body. The ignition assembly may be positioned adjacent to a combustible layer, e.g., diazodinitrophenel (DDNP) and nitrocellulose. Alternatively, the combustible layer may include a gas-generating layer, e.g., black powder, smokeless powder or a small amount of explosive or initiator such as tricinate, DDNP, lead azide, etc.

Once the elongate body has been desirably directed adjacent to or against a region of tissue, the ignition assembly may be actuated to ignite the combustible layer thereby resulting in an explosion. The resulting shock wave and/or expanding gas impinges against the proximal surface of a carriage which is thereby pushed distally at high speed until the distal surface of the carriage is pushed into contact against a stop or annular retaining lip and the high-impulse is imparted to the tissue anchor which is ejected at high-speed sufficient to penetrate into the tissue.

Other examples of mechanisms for creating a high-impulse shock wave include vaporizing fluid within the launch assembly to result in a rapidly expanding gas which may impart a high-impulse upon the anchor. Other variations may include use of a distensible or flexible membrane positioned distally of the fluid and against the proximal surface of the carriage for imparting the impulse against the carriage. Yet another example may include a hydraulically linked piston through the elongate body. Another variation may also include a pulsed laser for vaporizing a small portion of the carriage surface which results in a shock wave which propels the carriage distally to launch the anchor. Yet another variation may include a compression spring which may be used to store potential energy which may then be released to impart a high-impulse to the tissue anchor. And another variation may also include an electromagnetic assembly which may be activated to launch a magnet on the carriage having an opposite polarity.

An optional biasing element or spring may also be incorporated within the launch assembly to impart a returning or restoring force to the carriage such that after the anchor has been launched, the spring acts to urge the carriage proximally back to its initial position, where it may be launched again.

### BRIEF DESCRIPTION OF THE DRAWINGS

FIG. 1 shows an assembly view of a high-impulse anchor delivery system which is configured to launch tissue anchors intravascularly and/or intraluminally.

FIGS. 2A and 2B illustrate one variation of a spark-generating assembly with a combustible layer for creating a high-impulse shock wave to launch tissue anchors.

FIGS. 3A and 3B illustrate another variation of a spark-generating assembly for vaporizing a fluid for creating the high-impulse shock wave.

FIGS. 4A and 4B illustrate another variation for vaporizing a fluid and venting the gas.

FIGS. 5A and 5B show another variation for creating a high-impulse shock wave which is contained by a distensible or flexible membrane.

FIGS. 6A and 6B show another variation for creating a high-impulse anchor launching assembly utilizing hydraulic pressure transmitted through the elongate body.

FIGS. 7A and 7B illustrate another variation where laser energy may be used to vaporize a portion of the anchor launch carriage to create a high-impulse shock wave.

FIGS. 8A to 8C illustrate yet another variation where potential energy stored by a biasing element such as a compression spring may be used to generate the high-impulse energy for launching a tissue anchor.

FIGS. 9A and 9B illustrate yet another variation where an electromagnet may be used to impart the high-impulse energy to launch the tissue anchor.

FIGS. 10A to 10C show side, front, and back end views, respectively, of one variation of a tissue anchor.

FIGS. 11A and 11B illustrate barbed tissue anchors and anchors having pivotable barb members, respectively.

FIG. 12 illustrates a tissue anchor variation having an eyelet for passing a suture or tensioning element therethrough.

FIGS. 13A and 13B show a biased anchor variation having two piercing and anchoring tips connected via a curved connecting element in a first delivery configuration and a second anchoring configuration, respectively.

FIGS. 14A and 14B illustrate one example for use where the tissue anchor delivery system may be advanced intravascularly for deploying one or more tissue anchors within a heart chamber of a patient.

FIG. 15 shows an example of a flexible biopsy gun assembly which may utilize any of the high-impulse firing mechanisms described herein.

FIGS. 16A to 16D illustrate one method of use for obtaining a biopsy tissue sample with the flexible biopsy gun assembly utilizing a high-impulse firing mechanism.

### DETAILED DESCRIPTION OF THE INVENTION

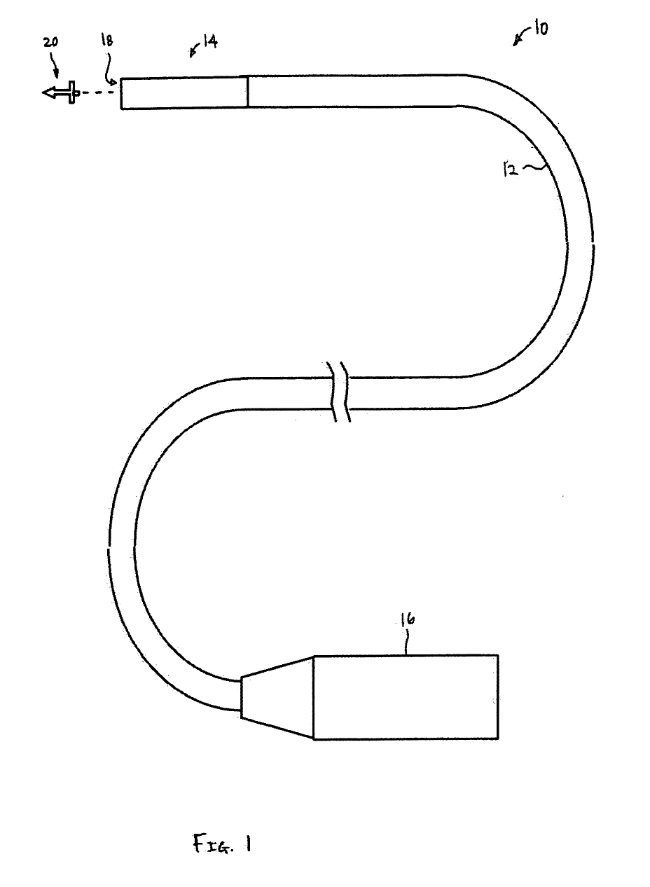

One example of an anchor delivery assembly 10 is shown in the assembly view of FIG. 1. The assembly 10 may generally have a handle assembly 16 for manipulation by a practitioner from outside the patient with a flexible elongate body 12 extending from the handle 16. The flexible elongate body 12 is generally a flexible tubular shaft which may be fabricated utilizing any number of catheter-based technologies provided that the elongate body 12 is sufficiently flexible to be advanced within the patient through the vasculature or intraluminally.

An anchor launch assembly 14 may be positioned upon the distal end of the elongate body 12 and may house one or more tissue anchors 20 which may be deployed or ejected at high speed through distal opening 18 defined in the distal end of the anchor launch assembly 14. The one or more tissue anchors 20 may be ejected by manipulating a control mechanism located on handle 16 from outside the patient body.

In one example of use, the anchor launch assembly 14 and elongate body 12 may be introduced percutaneously through an incision, e.g., through a femoral or jugular vein, and advanced through the patient's vasculature, e.g., through the inferior or superior vena cava, until access to the patient's ventricular heart chamber is acquired. Once within the patient's heart, the anchor launch assembly 14 may be directed to a region of cardiac tissue to be treated, for example, by steering the distal end of the catheter 12. Once the distal opening 18 is positioned directly adjacent to or against the desired tissue region, the one or more tissue anchors 20 may be launched at high speed from the anchor launch assembly 14 and into the tissue region.

Despite the low mass of the tissue anchor 20 relative to the underlying tissue resistance and also despite the inherent flexibility of the elongate body 12, a tissue anchor launched rapidly from the launch assembly 14 within a short launch time will generate high kinetic energy and may permit the generation of a high-velocity anchor having a large impulse to facilitate the insertion of the tissue anchor 20 into the underlying tissue.

A number of different mechanisms may be used to generate a high- velocity high-impulse anchor launch assembly 14. One example may utilize a spark generator positioned upon the end of a catheter for creating an explosive shock wave, typically used in intracorporeal lithotripsy instruments, as described in detail in U.S. Pat. No. 4,605,003 to Oinuma et al., which is incorporated herein by reference in its entirety.

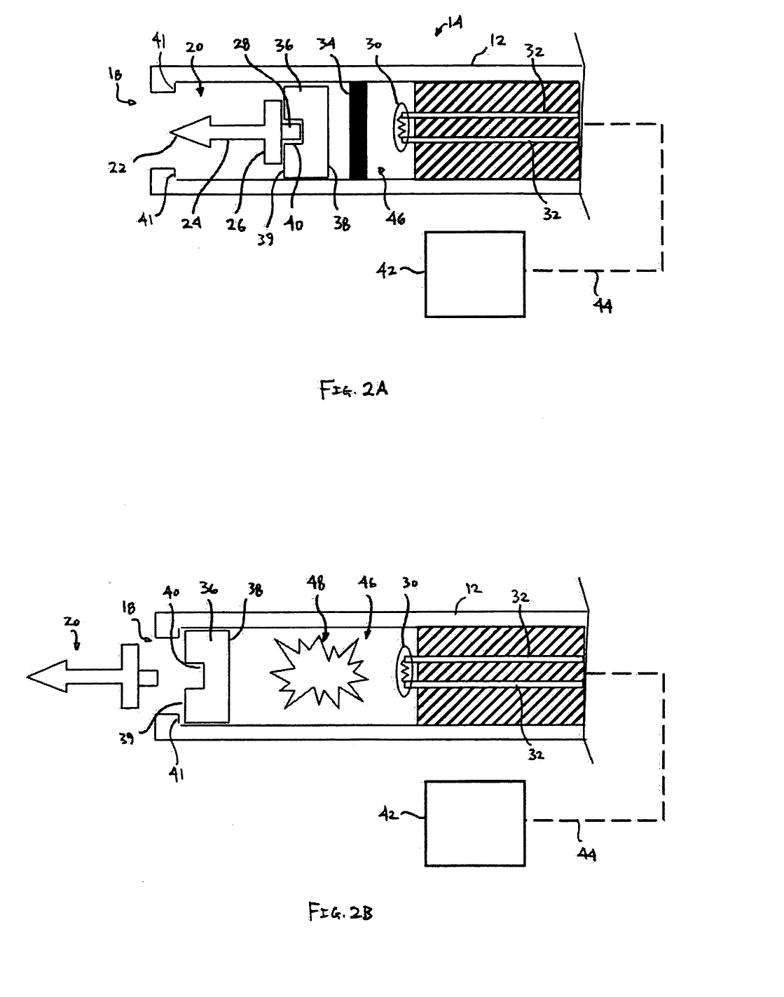

As shown in FIGS. 2A and 2B, a spark generating assembly may be utilized to create a high-impulse shock wave to drive a tissue anchor from the launch assembly 14. As shown in the side view of the launch assembly 14, spark ignition assembly 30 may be connected via one or more wires 32 routed through elongate body 12 to an ignition system or power supply 42 located outside the patient body. The power supply 42 may be connected to elongate body 12 via cable 44. Ignition assembly 30 may be positioned within an ignition chamber 46 adjacent to a combustible layer 34. The combustible layer 34 may include a number of different explosive materials, e.g., diazodinitrophenel (DDNP) and nitrocellulose. Alternatively, the combustible layer 34 may include a gas-generating layer, e.g., black powder, smokeless powder or a small amount of explosive or initiator such as tricinate, DDNP, lead azide, etc.

A ram or carriage 36 may be positioned distal to the combustible layer 34 within launch assembly 14 and may be made from any number of materials, e.g., brass, stainless steel, nickel, etc., provided that the material is sufficiently strong enough to withstand an explosive impact. Carriage 36 is configured to have a proximal or first surface 38 facing the combustible layer 34 and a distal or second surface 39 which defines an anchor engagement groove or slot 40.

The tissue anchor 20 may include a projection or locking member 28 which seats within the groove or slot 40 to maintain an orientation of tissue anchor 20 during delivery and launch. Tissue anchor 20 may further include a shank 24 which extends linearly to a piercing tip 22. A tissue stop 26, which may simply include one or more radial projections or a portion of shank 24 having a relatively larger diameter, may be optionally included along the anchor 20 to limit the depth to which the piercing tip 22 of anchor 20 is driven into the underlying tissue when launched from launch assembly 14. A suture may also be attached to the proximal tail of the anchor for attachment to another structure.

As shown in FIG. 2B, once the elongate body 12 has been desirably directed adjacent to or against a region of tissue, ignition assembly 30 may be actuated to ignite the combustible layer 34 thereby resulting in an explosion 48 within ignition chamber 46. The resulting shock wave and/or expanding gas is confined within the ignition chamber 46 and impinges against the proximal surface 38 of carriage 36, which is thereby pushed distally at high speed such that carriage 36 is translated within launch assembly 14 until the distal surface 39 is pushed into contact against a stop or annular retaining lip 41 defined at the distal opening 18. Once carriage 36 has been stopped, the high-impulse from the carriage 36 may be imparted to the tissue anchor 20 which unseats from groove or slot 40 and is ejected through distal opening 18 at high-speed sufficient to penetrate into the tissue.

Another example for creating a high-impulse shock wave is shown in FIG. 3A where an expandable fluid or gas 50 may be filled within ignition chamber 46 by a fluid delivery tube 52 having a tube opening 54 within chamber 46. Fluid 50, such as water or saline (or a combustible gas) may be pumped into chamber 46 via a fluid reservoir and pump assembly 56 connected via feed line 58 to elongate body 12. Once the chamber 46 has been sufficiently filled, ignition assembly 30 may create a spark to vaporize at least part of the fluid 50 contained within the chamber 46. As shown in FIG. 3B, the resulting vaporized fluid 48 may expand rapidly thereby pushing upon the proximal surface 38 of carriage 36 and imparting a high-impulse upon the anchor 20. As the carriage 36 is accelerated rapidly, anchor 20 may be launched from assembly 14, as described above.

In one variation, the spark generator 42 and ignition assembly 30 may be configured to produce an output pulse of up to several microseconds at several thousand volts with a current of up to 1 thousand amperes. Examples of spark generators for vaporizing contained fluids is shown and described in further detail in U.S. Pat. No. 5,281,231 to Rosen et al., which is incorporated herein by reference in its entirety.

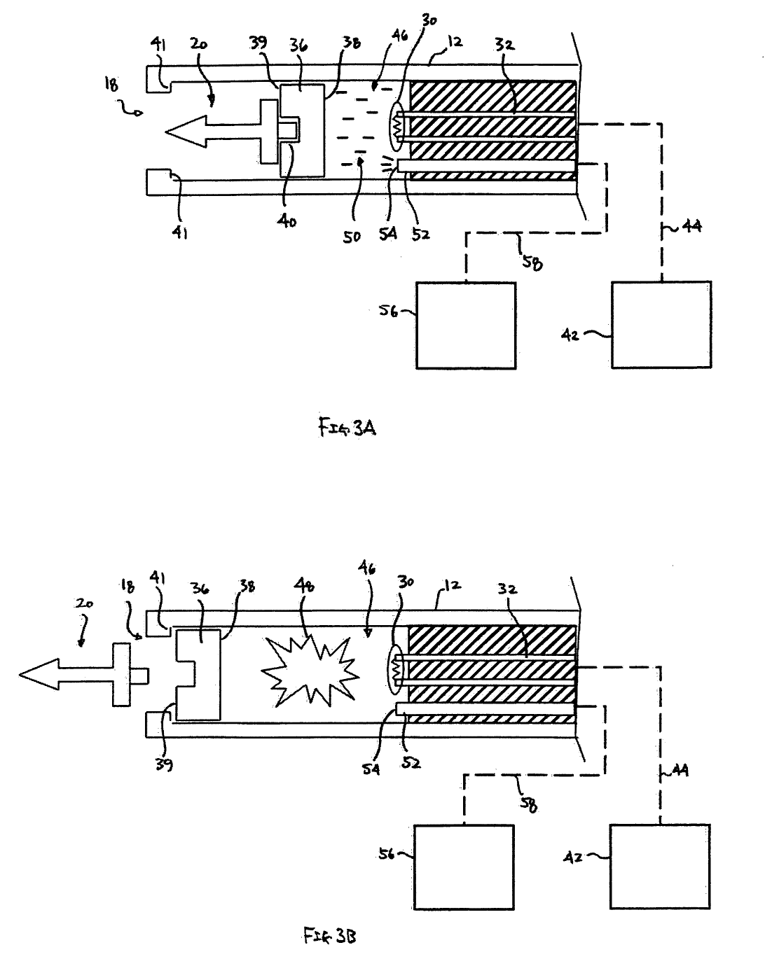

In another similar variation, one or more openings 60 may defined through the wall of launch assembly 14 as shown in FIG. 4A. When carriage 36 is positioned proximally in its pre-ignition position, carriage 36 may cover the openings 60 to prevent fluid from escaping. Once the fluid 50 has been vaporized and expands, carriage 36 may expose the openings 60 as it translates distally through launch assembly 15. As the vaporized fluid pushes against carriage 36, the gas may be at least partially vented 62 to control the amount of impulse imparted against carriage 36, as shown in FIG. 4B. The number and positioning of openings 60 may be varied depending upon the desired degree of force transfer to carriage 36 and anchor 20.

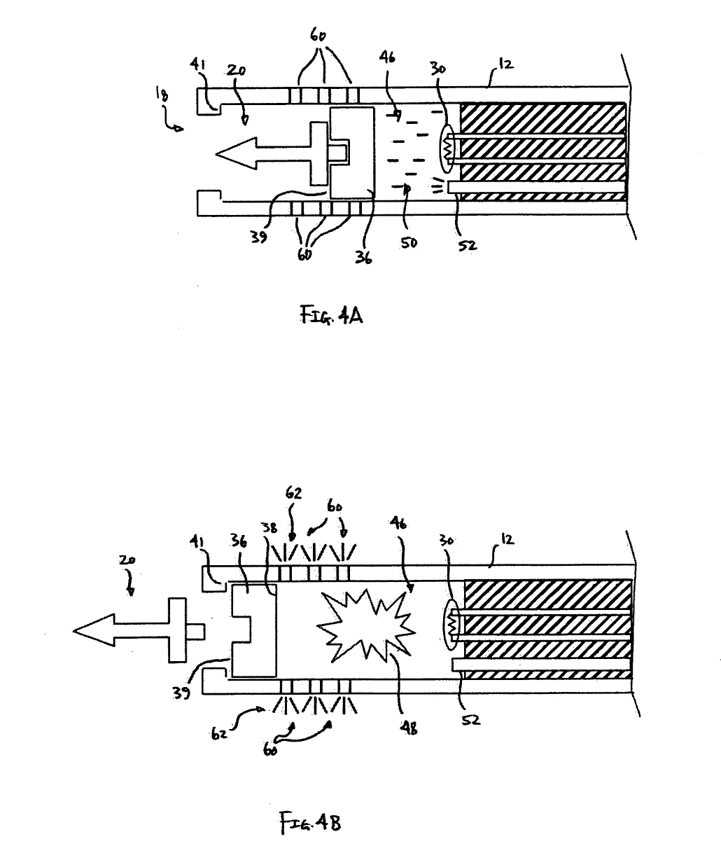

Aside from utilizing vents, the launch assembly 14 may alternatively use a distensible or flexible membrane 64 positioned distally of the fluid 50 and against the proximal surface 38 of carriage 36, as shown in the variation of FIG. 5A. The membrane 64 may be comprised of any number of distensible or flexible polymeric materials. When fluid 50 has been vaporized, the expanding vapor or gas 48 may impart an impulse against membrane 64 which may then distend and impart the impulse against proximal surface 38 to launch carriage 36 and anchor 20, as shown in FIG. 5B. Membrane 64 may eventually flex back into its original shape while containing the vaporized fluid within ignition chamber 46.

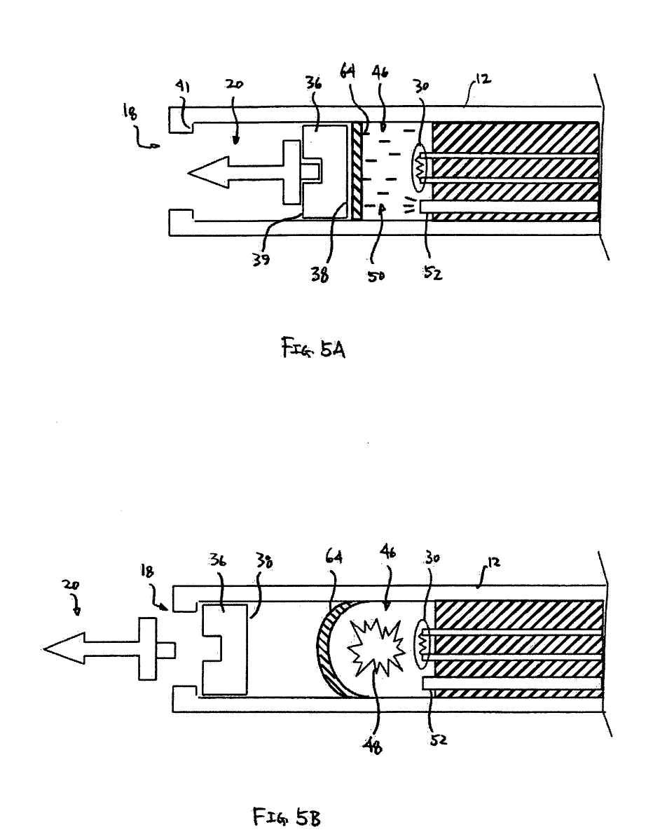

Aside from the use of explosives and spark generators, another variation may utilize hydraulic pressure to impart a high-impulse for launching tissue anchors into underlying tissue. FIG. 6A shows one example in which a fluid delivery tube 70 may extend in fluid communication from a hydraulic carriage 72 positioned near the distal end of elongate body 12, through the length of elongate body 12, and proximally to a fluid reservoir 80. The hydraulic carriage 72 may be positioned translatably over a distal portion of tube 70 such that the carriage 72 at least partially surrounds the tube 70 within a variable fluid chamber 74. A gasket or seal 76 may be positioned between an outer surface of the surrounded tube 70 and an inner surface of the variable fluid chamber 74 to inhibit the leakage of fluids therebetween. A tissue anchor 20 may be seated against a distal surface 39 of the hydraulic carriage 72.

A ram 82 may be urged distally, mechanically or electrically, from a proximal end of elongate body 12 to press against the fluid contained within the reservoir 80, as shown in FIG. 6B. The fluid may be an incompressible fluid, such as water or saline or a perfluorohydrocarbon fluid such as Fluorinert® (3M Corporation). As the fluid is urged distally through elongate body 12, the fluid 84 may be forced through tube 70 to exit opening 78 and fill fluid chamber 74. As the fluid 84 continues to fill chamber 74, hydraulic carriage 72 may be urged to translate distally, much like a hydraulic piston, until distal surface 39 contacts stop 41 and anchor 20 is launched. The force imparted to ram 82 may be pulsed rapidly such that the impulse transferred via the fluid through elongate body 12 and to the anchor 20 via carriage 72 is rapid. Once the distal surface rests against stop 41, a negative pressure may be applied in the proximal end of the device to urge the carriage 72 proximally back to its original position.

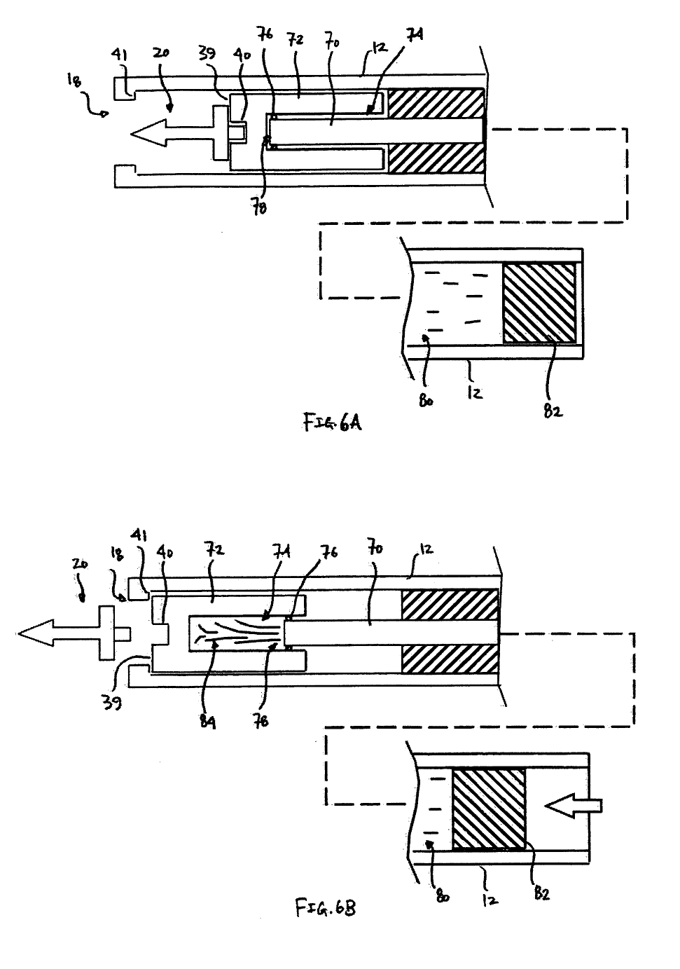

In yet another variation, a pulsed laser may be used to impart a shock wave to the anchor 20. The example shown in FIG. 7A illustrates an optical fiber 90 which may be positioned through elongate body 12 and connected to a laser generator. The fiber distal end 92 may be positioned immediately adjacent to the proximal surface 38 of carriage 36. In use, the laser generator may be activated such that the laser light 96 emitted from distal end 92 is incident upon the proximal surface 38 such that the carriage 36 absorbs the emitted laser light 96. The absorbed laser energy may vaporize a small portion of the carriage surface 98 such that the rapid vaporization results in a shock wave which propels the carriage 36 distally to launch the anchor 20, as shown in FIG. 7B. The proximal portion of carriage 36 may also contain a liquid such as water which may be vaporized to generate the shock wave.

The laser may be pulsed, e.g., at 1 microsecond durations at an energy level of 50 millijoule. Examples of pulsed laser generators imparting an impulse are described in further detail in U.S. Pat. No. 5,281,231 to Rosen et al., which has been incorporated by reference herein above.

An optional biasing element or spring 94 may also be incorporated by positioning the spring 94 within the launch assembly such that a distal part of the spring 94 is connected 100 to carriage 36 and a proximal part of the spring 94 is connected to a portion of the elongate body 12. When carriage 36 is adjacent to the optical fiber 90, spring 94 may be in a neutral or partially tensioned state, as in FIG. 7A. As carriage 36 is propelled distally, spring 94 may act to impart a returning or restoring force to carriage 36, as shown in FIG. 7B, such that after anchor 20 has been launched, spring 94 acts to urge carriage 36 proximally back to its initial position, where it may be launched again.

The use of a biasing element or spring 94 may be incorporated in any of the variations described herein, as practicable, to impart a returning or restoring force to the carriage, if so desired.

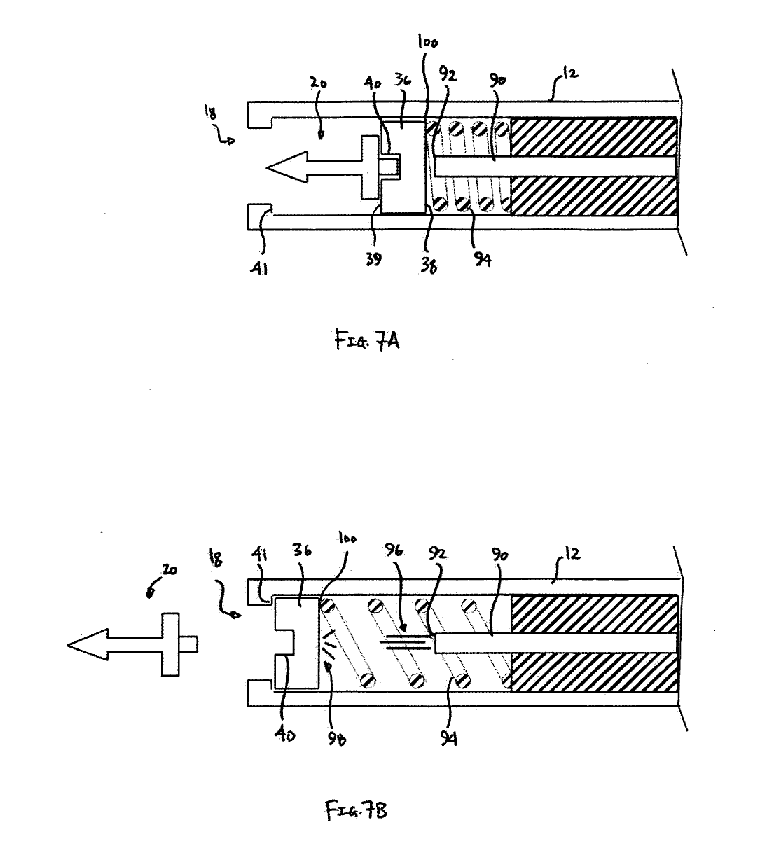

In another variation utilizing a spring element, FIG. 8A shows a compression spring 110 positioned within a compression chamber 112 between piston 114 and carriage 36 locked in place via one or more locking projections 118. Pusher 116 may be actuated from outside the patient body to push piston 114 distally such that compression spring 110 is compressed between piston 114 and proximal surface 38 of carriage 36. When compression spring 110 has been fully compressed, locking projections 118 may be moved from a locked position where projections 118 are positioned within one or more complementary retaining grooves 122 defined in carriage 36 to an unlocked position where the projections 118 are released from grooves 122 and moved into position within receiving grooves 120 defined within the launch assembly, as shown in FIG. 8B. Projections 118 may be actuated from the handle assembly 16 from outside the patient body. Once the projections 118 have been released from their locking configuration, carriage 36 is free to move distally urged at a high velocity by the released potential energy stored within compression spring 110 to launch anchor 20, as shown in FIG. 8C.

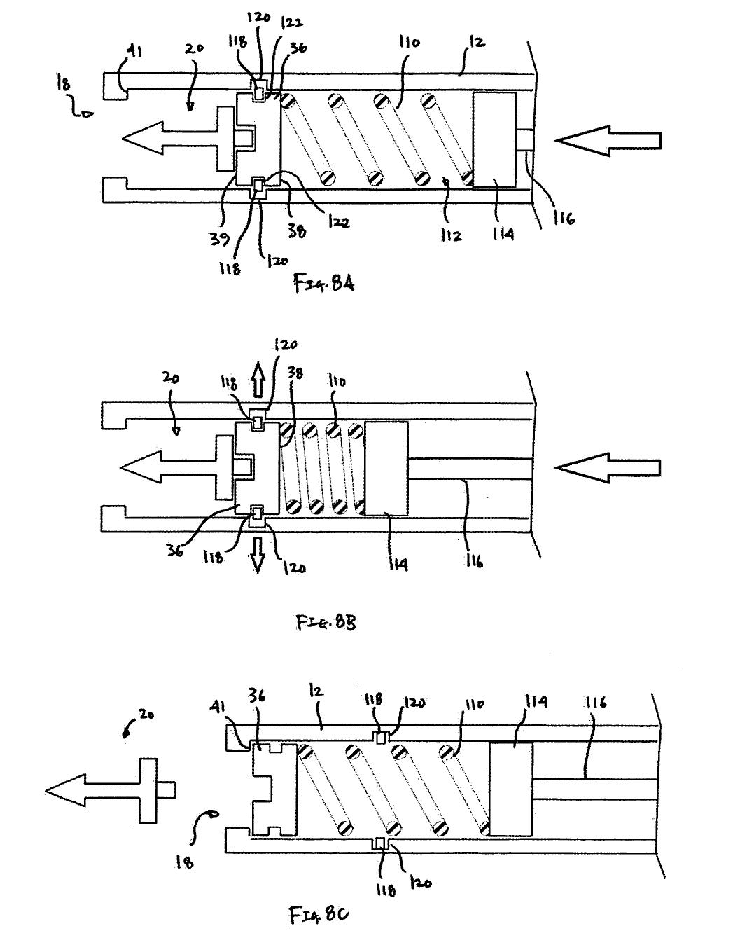

In yet another variation, carriage 36 may be launched via an electromagnetic assembly, as shown in FIG. 9A. As illustrated, carriage 36 may have a ferromagnet 130 attached to its proximal surface. An electromagnet 132 may be positioned within the launch assembly and connected via one or more electrical wires 134 to a power supply 136. The power supply 136 may be connected via a cable 140 to a controller 138 for actuating and/or controlling the power supply 136.

In use, as shown in FIG. 9B, power may be supplied to electromagnet 132 to generate a pulsed magnetic force opposite in polarity to the ferromagnet 130 positioned upon carriage 36. The pulsed magnetic force may thus repel ferromagnet 130 thereby launching carriage 36 and anchor 20. The power may be simply cycled down or a magnetic force having an opposite polarity may be generated by electromagnet 132 to urge the carriage 36 proximally to its initial position, where it may be launched again.

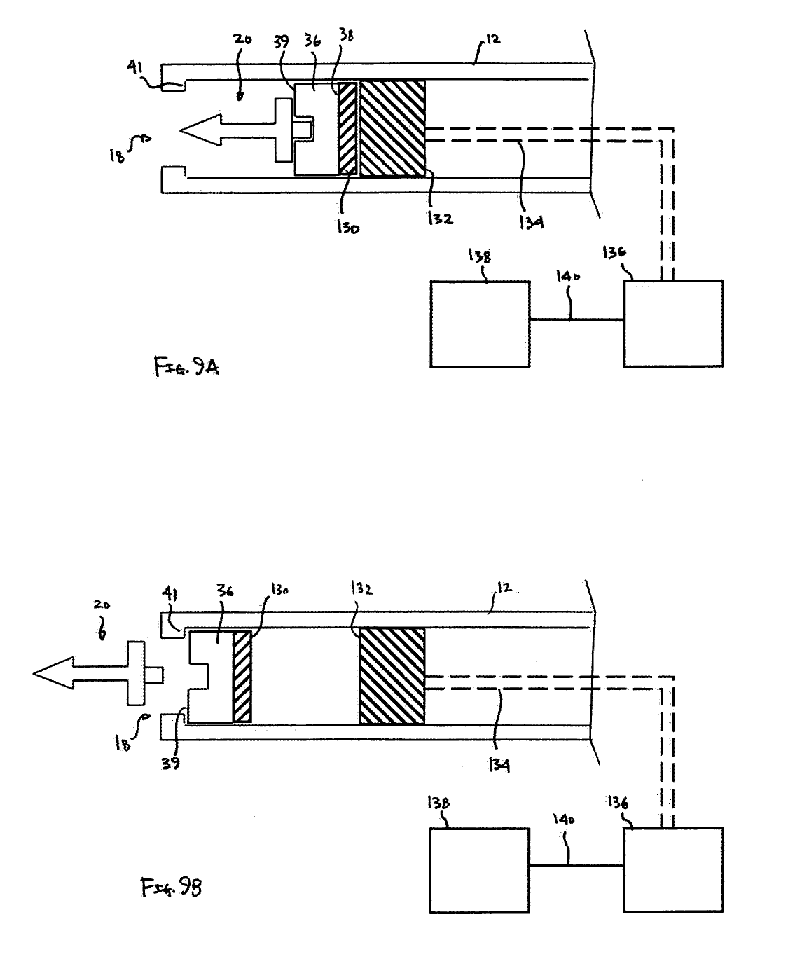

Turning now to the tissue anchors, various configurations may be utilized in combination with any of the anchor launching instruments described above. FIGS. 10A to 10C show side, front, and back end views, respectively, of one variation of a tissue anchor. As previously described, anchor 20 may include a shank 24 having a tapered or piercing tip 22 at a first end and a tissue stop 26 at a second end of the anchor 20. The seating projection 28 may extend proximally from the tissue stop 26.

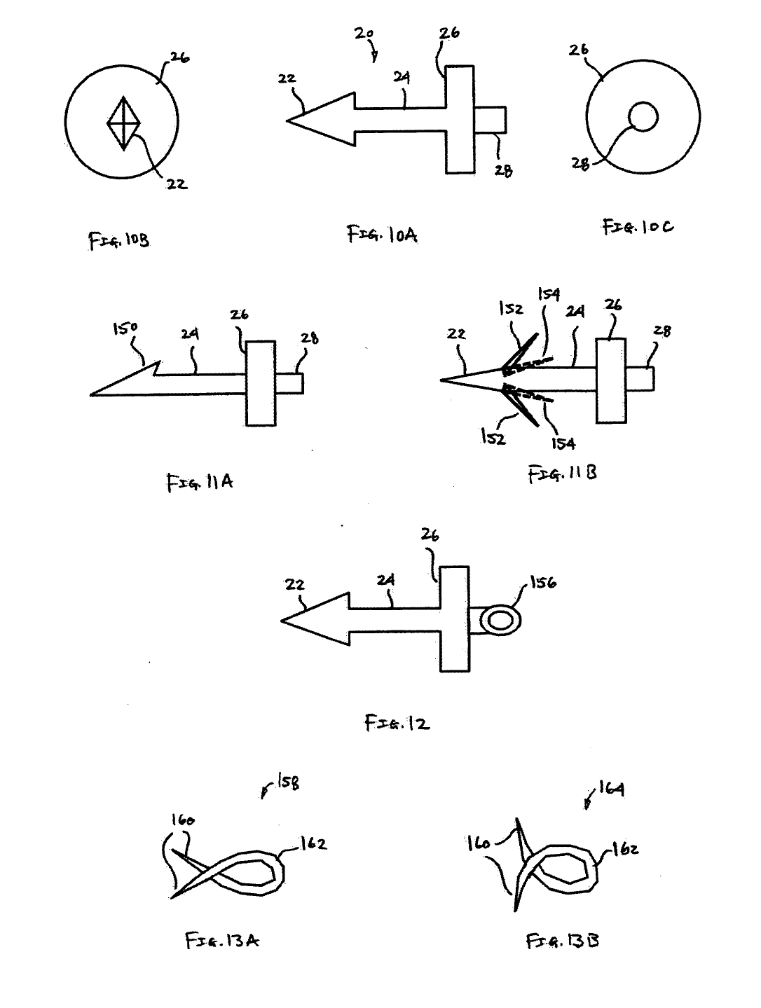

Another variation may include a tissue anchor having a barbed piercing tip 150 to inhibit the proximal withdrawal of the anchor from the tissue, as shown in FIG. 11A. Alternatively, one or more barbs near the piercing tip 22 may be pivotably connected to the anchor, as shown in FIG. 11B, such that during insertion into the tissue, the barbs 152 are configured into a low-profile deployment configuration 154 and when the anchor is fully seated within the tissue, the pivoting barbs 152 may project radially to further inhibit the proximal withdrawal of the anchor.

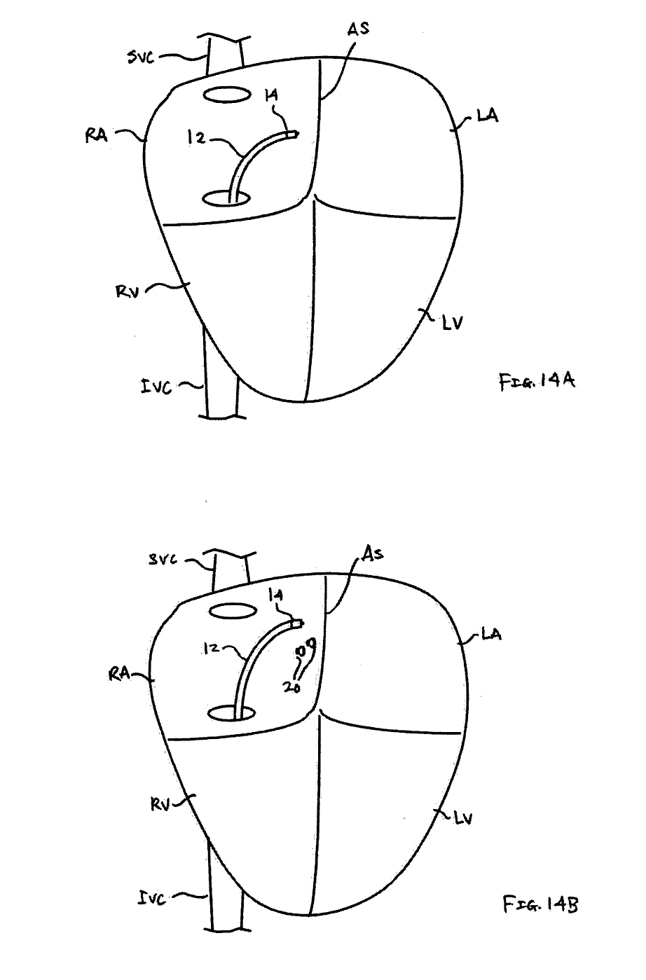

In another variation, the proximal projection may define an eyelet 156, as shown in FIG. 12, to allow for the passage of a suture or other tensioning element therethrough. Another variation for tissue anchors is shown in FIG. 13A, which shows a biased anchor 158 having a first deployment configuration where two piercing and anchoring tips 160 are connected via a curved connecting element 162. When deployed into the tissue, curved connecting element 162 may reconfigure itself into a relaxed anchoring configuration 164 where the anchoring tips 160 are projected into an expanded shape for inhibiting withdrawal from tissue.

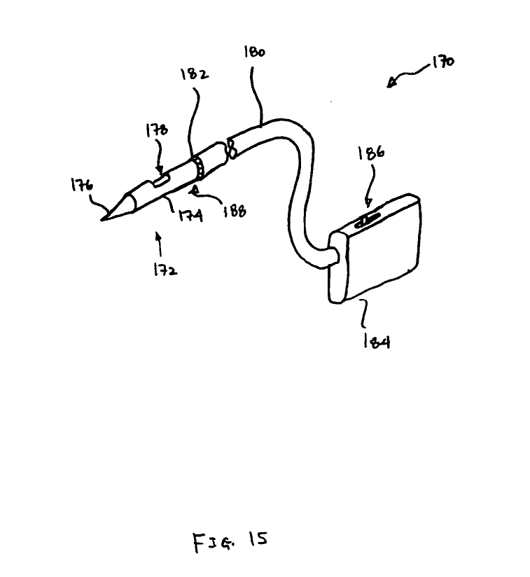

In an example of one method for using the anchor delivery assembly, FIGS. 14A and 14B show how the elongate body 12 and anchor launch assembly 14 may be advanced intravascularly, e.g., through the inferior vena cava IVC and into the right atrium RA of a patient. Other anatomical landmarks including the superior vena cava SVC, left atrium LA, and left and right ventricles LV, RV are also shown. With anchor launch assembly 14 positioned within the right atrium RA, elongate body 12 may be articulated and/or steered to direct the launch assembly 14 to a region of tissue, such as the atrial septum AS.

With launch assembly 14 desirably placed adjacent to the tissue surface, one or more tissue anchors 20 may be deployed utilizing any of the mechanisms described here. Several tissue anchors 20 may be deployed around the tissue by deploying a first anchor, repositioning the launch assembly 14 and deploying a second anchor, and so on, until a desired number of anchors have been placed into the tissue. Such an instrument may be utilized for procedures including the closure of atrial septal defects, closure of patent foramen ovale, and any number of other indications.

In another application of the mechanisms described herein, FIG. 15 shows an assembly view of biopsy gun assembly 170 which may utilize any of the high-impulse mechanisms. In this assembly 170, a coring needle 174 having a hollow shaft and piercing tip 176 may be translatably positioned within a lumen 188 of an elongate and flexible member 180 which is sufficiently sized for intravascular or endoluminal advancement within a patient body.

The coring needle 174 may define at least one opening 178 along its side for collecting biopsy tissue samples within. The distal end of elongate flexible body 180 may have a tapered or cutting edge 182 while the proximal end may be connected or attached to a handle assembly 184 which may be manipulated from outside the patient body. A firing control 186 may be integrated within handle 184 for rapidly launching the coring needle 174 and/or elongate body 180 during tissue biopsy procedures. The high-impulse mechanism may be positioned proximal to coring needle 174 within elongate body 180 or within handle 184 depending upon the type of mechanism is utilized to create the shock wave.

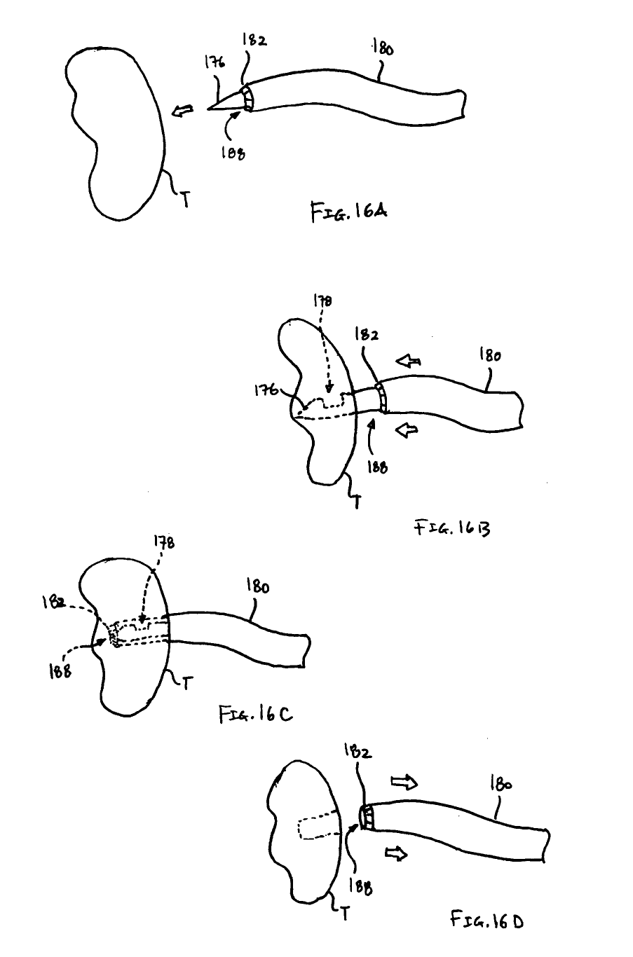

In one example of use, the elongate flexible body 180 may be advanced or steered intravascularly or endoluminally to position the distal end adjacent to a tissue region T from which a tissue sample is to extracted, as shown in FIG. 16A. Coring needle 174 may be readied to be launched and then fired into the tissue T until piercing tip 176 and opening 178 has penetrated into the tissue, as shown in FIG. 16B. If opening 178 has not been introduced within the tissue T, coring needle 174 may be retracted and then re-fired by the impinging shock wave.

Once opening 178 has been sufficiently buried within the tissue T, elongate body 180 may then be advanced distally along coring needle 174 to allow cutting edge 182 to slide over opening 178, thereby cutting and capturing any tissue which may have entered opening 178 much like a guillotine, as shown in FIG. 16C. Alternatively, coring needle 174 may be retracted back into lumen 188 while cutting any tissue trapped within opening 178. With a tissue sample secured within opening 178, both coring needle 174 and elongate body 180 may be withdrawn from tissue T, as shown in FIG. 16D, and removed from the body to extract the captured tissue for analysis.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue anchor launching mechanism, comprising:
a high-impulse energy generating assembly configured to generate an impinging shock wave; and
one or more tissue anchors configured to be propelled via the shock wave such that the tissue anchor is ejected into a tissue region.

2. The mechanism of claim 1 further comprising a carriage having a first surface for contact against the impinging shock wave generated by the energy generating assembly and a second surface for pushing against the one or more tissue anchors, wherein the carriage is sized for intravascular and/or intraluminal delivery through a patient body.

3. The mechanism of claim 2 further comprising an elongate body having a flexible length, wherein the energy generating assembly and carriage are positionable within a distal end of the elongate body.

4. The mechanism of claim 3 further comprising a handle assembly connected to a proximal end of the elongate body.

5. The mechanism of claim 1 further comprising a plurality tissue anchors adapted for delivery into or against the tissue region.

6. The mechanism of claim 1 wherein the energy generating assembly comprises an ignition assembly.

7. The mechanism of claim 6 further comprising a combustible material positioned between the ignition assembly and the first surface of the carriage.

8. The mechanism of claim 7 wherein the combustible material comprises diazodinitrophenel, nitrocellulose, black powder, smokeless powder, tricinate, or lead azide.

9. The mechanism of claim 6 further comprising a vaporizable fluid between the ignition assembly and the first surface of the carriage.

10. The mechanism of claim 9 further comprising a distensible or flexible membrane positioned between the vaporizable fluid and the first surface of the carriage.

11. The mechanism of claim 1 wherein the energy generating assembly comprises a hydraulic piston in fluid communication with a ram positioned outside the patient body.

12. The mechanism of claim 2 wherein the energy generating assembly comprises an optical fiber which is positioned proximal to the carriage to enable laser energy transmitted therethrough to be incident upon the first surface of the carriage.

13. The mechanism of claim 2 wherein the energy generating assembly comprises a compression spring positioned against the first surface of the carriage and a piston proximal to the compression spring for compressing the spring.

14. The mechanism of claim 2 wherein the energy generating assembly comprises an electromagnet positioned proximal to the carriage, wherein the carriage further comprises a magnet attached thereto and having a polarity opposite to a polarity of the electromagnet.

15. A method for intravascularly and/or intraluminally launching a tissue anchor having a high-impulse, comprising:
advancing the tissue anchor intravascularly and/or intraluminally;
positioning the tissue anchor relative to a tissue region to be treated; and
generating a high-impulse energy proximal to the tissue anchor such that the tissue anchor is launched at a high-velocity into the tissue region.

16. The method of claim 15 wherein advancing the tissue anchor comprises advancing intravascularly into a chamber of a heart.

17. The method of claim 15 wherein positioning the tissue anchor comprises steering an anchor launching assembly within which the tissue anchor is disposed relative to the tissue region.

18. The method of claim 15 wherein generating a high-impulse energy comprises creating an explosive shock wave proximal to a translatable carriage upon which the tissue anchor is positioned.

19. The method of claim 18 wherein creating an explosive shock wave comprises exploding a combustible material via an ignition assembly.

20. The method of claim 18 wherein creating an explosive shock wave comprises vaporizing a fluid via an ignition assembly.

21. The method of claim 18 wherein creating an explosive shock wave further comprises containing the shock wave within a distensible or flexible membrane.

22. The method of claim 18 wherein creating an explosive shock wave comprises transmitting laser energy via an optical fiber to vaporize a portion of a proximal surface of a translatable carriage upon which the tissue anchor is positioned.

23. The method of claim 15 wherein generating a high-impulse energy comprises transmitting the energy via a hydraulic piston.

24. The method of claim 15 wherein generating a high-impulse energy comprises releasing a compression spring proximal to a translatable carriage upon which the tissue anchor is positioned.

25. A tissue anchor launching assembly, comprising:
an elongate body having a proximal end, a distal end, and a flexible length therebetween sized for intravascular and/or intraluminal delivery through a patient body
a high-impulse energy generating assembly disposed near or at the distal end; and
a carriage translatably positioned distal to the energy generating assembly, the carriage having a first surface for contact against an impinging shock wave generated by the energy generating assembly and a second surface for pushing against one or more tissue anchors.

26. The assembly of claim 25 further comprising a handle assembly connected to the proximal end of the elongate body.

27. The assembly of claim 25 wherein the energy generating assembly comprises an ignition assembly.

28. The mechanism of claim 27 further comprising a combustible material positioned between the ignition assembly and the first surface of the carriage.

29. The mechanism of claim 28 wherein the combustible material comprises diazodinitrophenel, nitrocellulose, black powder, smokeless powder, tricinate, or lead azide.

30. The mechanism of claim 27 further comprising a vaporizable fluid between the ignition assembly and the first surface of the carriage.

31. The mechanism of claim 30 further comprising a distensible or flexible membrane positioned between the vaporizable fluid and the first surface of the carriage.

32. The mechanism of claim 25 wherein the energy generating assembly comprises a hydraulic piston in fluid communication with a ram positioned outside the patient body.

33. The mechanism of claim 25 wherein the energy generating assembly comprises an optical fiber which is positioned proximal to the carriage to enable laser energy transmitted therethrough to be incident upon the first surface of the carriage.

34. The mechanism of claim 25 wherein the energy generating assembly comprises a compression spring positioned against the first surface of the carriage and a piston proximal to the compression spring for compressing the spring.

35. The mechanism of claim 25 wherein the energy generating assembly comprises an electromagnet positioned proximal to the carriage, wherein the carriage further comprises a magnet attached thereto and having a polarity opposite to a polarity of the electromagnet.

36. A tissue biopsy assembly, comprising:
an elongate flexible member defining a lumen therethrough;
a high-impulse energy generating assembly positioned within or proximal to the elongate flexible member and is configured to generate an impinging shock wave; and
a coring needle defining an opening along a side and translatably positioned within the elongate flexible member and distal to the energy generating assembly, the coring needle being configured to be propelled via the shock wave such that the coring needle is ejected into a tissue region.

37. The assembly of claim 36 further comprising a carriage having a first surface for contact against the impinging shock wave generated by the energy generating assembly and a second surface for pushing against the coring needle, wherein the carriage is sized for intravascular and/or intraluminal delivery through a patient body.

38. The assembly of claim 36 further comprising a handle assembly connected to a proximal end of the elongate flexible member.

39. The assembly of claim 36 wherein the distal end of the elongate flexible member is tapered to a cutting edge.
